(19) United States
(12) Patent Application Publication
(10) Pub. No.: US 2008/0058709 A1
(43) Pub. Date: Mar. 6, 2008

(54) METHOD AND APPARATUS THAT ASSOCIATE THE PARENTERAL INJECTION OF MEDICAL GRADE CARBON DIOXIDE (CO2) CONCOMITANTLY WITH THE APPLICATION OF INFRARED RADIATION FROM THERMAL AND/OR LIGHT SOURCES USING CONTROL BY MEANS OF CUTANEOUS AND/OR BODY THERMOMETRY

(76) Inventor: Mario Augusto Da Silva Freitas, Santo Andre (BR)

Correspondence Address: STITES & HARBISON PLLC, 1199 NORTH FAIRFAX STREET, SUITE 900, ALEXANDRIA, VA, 22314, US

(21) Appl. No.: 11/848,486

(22) Filed: Aug. 31, 2007

(30) Foreign Application Priority Data
Aug. 31, 2006 (BR) PI0604018-7

(51) Int. Cl. A61M 37/00

(52) U.S. Cl. 604/23

(57) ABSTRACT

A method and apparatus that associate a new parenteral injection of medical grade carbon dioxide concomitantly with the application of infrared radiation from thermal and/or light sources using control by means of cutaneous and/or body thermometry, associating the concomitant application of infrared radiation from different light or thermal sources, to the direct injections of medical grade carbon dioxide CO₂ into parenteral routes via carbon dioxide infusion regulating apparatuses and infrared radiation emitting apparatuses controlled by cutaneous and/or body thermometry, either direct or indirect.

### BACKGROUND OF THE INVENTION

The present invention refers to a method and apparatus that associate the concomitant application of infrared radiation produced by different thermal and/or light sources to direct injections of medical grade carbon dioxide (CO₂) in parenteral routes through the use of CO₂ infusion apparatus controlled by cutaneous thermometry. More precisely, the present method combines the benefit of the biological actions observed in the application of infrared radiation and the CO₂ in the different organic tissues. Such association is intended to augment the individual effects of each method by physical, chemical, and biological interactions, causing each method to act as an inducing, promoting, or potentiating agent of several organic effects as related to the other, particularly in analgesia during the processes of parenteral injection of the CO₂. The control of the procedures by body thermometry has the following objectives: to allow for imaging diagnosis (thermographies) or graphic thermometries of the affected areas, to guide the sites of CO₂ injection and infrared radiation application, to avoid complications of the procedure (e.g., pain, ischemia, skin burns), to allow variation in infrared radiation intensity and CO₂ infusion according to the requirements of the affected tissues, as well as to allow, in the form of a subsidiary test, the recording of comparative data prior to and following the described therapies.

### DESCRIPTION OF THE PRIOR ART

Basics of the Technique—Introduction

1—Physiology of the Circulatory System

All living beings depend on exchanges for their survival. In the intimacy of the cell, this mechanism enables the exchange of harmful substances and products of metabolism by other substances that are indispensable for organic reactions and the production of energy. The greater the phylogenetic evolution, the greater the complexity in organic exchange systems.

The circulatory system, in the organisms that have it, has the elemental function of optimizing said exchanges, adjusting them to the different body tissues (peripheral capillary diffusion, hematosis, glomerular filtration, hematoencephalic barrier, and other).

Anatomically, it consists of a propulsive organ (the heart), a vast network of efferent (the arteries) and afferent conduits (the veins), with a junction portion, composed of microscopic vessels (the capillaries). The vascular capillaries associated with the distal portion of afferent (arterioles) and efferent conduits (venules), and their intercommunications (shunts) constitute the microcirculation.

Presently, it is known that the innermost layer of the conducting circulatory systems, the endothelium, depending on the type of vessel (artery, vein, lymphatic vessel, or capillaries), shows differences in its morphological structure, as well as in its secretory functions. This is due to the specific properties of the different receptors on its cell surfaces, sensitive to various stimuli, such as: inflammatory substances, intravascular pressure, blood cell secretions, vascular growth factors, tissue growth factors, and hypoxia.

This vascular and perivascular microenvironment is responsible for a great amount of cell reactions, related both to hemodynamic control and to the normal tissue repair processes, such as cicatrization. It may also ensure the survival of normal tissues, such as skin grafts, or even the development of abnormal tissues, such as certain types of neoplasias.

The processes of tissue repair and the growing tissues have a high local metabolic demand, with a high consumption of energy, thus a greater need for oxygenation. In order to satisfy such process, the circulatory system makes use of two basic mechanisms, vasodilatation and neoangiogenesis.

Vasodilatation is dependent on the presence of smooth muscles on the vascular walls. It may happen on large caliber vessels, as well as at the origins of microcirculation (arterioles), temporarily increasing the blood flow to the site (hyperemia) within a limit pre-established by the dimensions of the vessels involved and their capacity to respond to local chemical changes.

On the other hand, neoangiogenesis is a much more complex process, involving the progression of the capillary network by direct growth, a process that definitely increases the blood flow at the site where it occurs. This mechanism provides the area involved with a lesser sensitivity to local chemical variations, maintaining higher baseline blood flow.

Several endogenous chemical substances have been shown to be capable of producing or inducing vasodilatation, such as: prostaglandins, bradykinins, lactic acid, nitric oxide, and carbon dioxide (CO₂).

The processes of neoangiogenesis appear to be related with cell growth factors produced by specific normal cells, such as vascular endothelial cells, fibroblasts, and other contributing factors, such as ovarian hormones and liver growth factors.

Certain substances have been described as capable of potentiating endothelial mitoses, such as the Vascular Endothelial Growth Factors VEGF 165 and VEGF 121, by inducing gene expressions. Several histological cell types of tumors are also related with the production of factors that stimulate neoangiogenesis.

Reactions and stimuli triggered by local factors, such as lack of tissue oxygenation, also appear to have such property, by means of specific receptors that stimulate the production of Hypoxia Induced Factors (HIF 1α and HIF 1β), which are likely responsible for gene stimulation and expression, for increasing Vascular Endothelial Growth Factor secretion, and the development of a collateral circulatory network.

Based on the above mentioned scientific knowledge, CO₂ applied directly to tissues can mimic tissue hypoxia or momentarily augment it, causing no effective harm to tissue, acting by stimulating specific receptors or providing the release of tissue or vascular factors that induce or promote local neoangiogenesis.

Despite the observation of some clinical effects, familiarity of the mechanisms of action of CO₂ directly on tissues is still unknown, and it could be potentiated by other means, such as, for instance, infrared radiation.

2—Applied Pharmacology

The great amount of tissues capable of cell repair and the several ways that these may respond to stimuli for angiogenesis, leave this field open for the development of new technologies and for the treatment of several diseases related to microcirculation.

For the therapeutic effects of a certain drug to occur in the human body, such drug or its byproducts are required to contact the target cells directly, interacting with their cell membranes.

Because our inner medium is isolated from the external by cutaneous and mucosal barriers, in order to reach certain specific locations in the body, we need to make use of administration routes for the drugs, so as to make them cross these barriers and enter into organic tissues. This, in our current pharmaceutical knowledge, can be achieved by two basic routes: the enteral and the parenteral route.

Enteral routes are those in which the drug is absorbed at some point in the digestive system, makes its way into the hepatic portal venous system and reaches the liver in great amounts, an environment liable to several metabolic processes by hepatocytes.

In the event, however, that primary liver metabolism is not desired, or a direct action is required at the site of drug application, the parenteral routes are chosen.

There are multiple known parenteral routes, however, those that represent a target for possible therapeutic applications of medical grade carbonic anhydride (CO₂) are: transcutaneous, intralesional, intradermal, subcutaneous, perifascial, intramuscular, peritendinous, periarticular, intrasynovial, perivascular, intravascular, retroperitoneal, peridural, subdural, and perineural.

3—Carbon Dioxide

Carbon dioxide (CO₂) is a gas present in Earth's atmosphere and in living organisms, both plant and animal. It may also be referred to as carbonic anhydride, carbonic acid gas, or carbon bioxide. It is a non toxic, non oxidative, non allergenic, non embolic gas highly diffusible in lipoprotein membranes.

This gas is found as a product of normal cell metabolism following intracellular chemical oxidative reactions with expenditure of energy. Its elimination from cells is by passive transport through the cell membrane, and it is removed from any point of the human body by the capillary network stemming from a normal circulation.

Its elimination from the human body is mainly achieved through breathing, by means of direct diffusion in the alveolar capillaries. Large volumes of carbon dioxide can safely be eliminated through this route.

In the presence of tissue hypoxia (low levels of oxygenation in tissues), the amount of autologous CO₂ production is increased. In cases of ischemia (low levels of local blood flow), its removal is extremely delayed.

Accordingly, there is a local accumulation of the gas in poorly oxygenated and poorly vascularized tissues, which could probably be associated with the stimulation of hypoxia induced receptors (HIF) or even the indirect induction of secretion of Vascular Endothelial Growth Factors and Tissue Growth Factors.

In nature, CO₂ is found as a highly important gas in maintaining the biosphere and the planet's thermal balance. It is absorbed by most plants and algae as an essential element for the process of cell respiration.

From all the gases present in the Earth's atmosphere, carbon dioxide is the one that appears to have the greatest capacity to absorb solar infrared radiation, retaining the heat that it carries, and maintaining atmospheric heat, which is essential to all forms of life on the planet.

Due to the great amount of emission of pollutants, burned over lands, and combustion products from motor vehicles, there is a progressive increase in the concentration of atmospheric CO₂ in several parts of the world, which contributes with the increased environmental temperature in these places, a process also referred to as the “greenhouse effect”.

The effects of warming of the Earth's atmosphere, observed after the solar infrared radiation is absorbed by carbon dioxide (CO₂), since there is no structural change made to the gas when it is used as a drug, could be reproduced in the human body, especially on the sites and tissues where it would be injected, as is the case with the applications of the gas via parenteral routes.

4—Infrared Radiation

Infrared radiation is a form of electromagnetic radiation produced by bodies heated as of 10 (ten) degrees Kelvin. The greater the heating and molecular agitation of bodies, the greater the possibility of emitting this radiation. Thus, several elements or apparatuses capable of producing thermal and light emissions are also regarded as sources of infrared radiation.

As with all electromagnetic radiation, however, infrared radiation may appear in different wavelengths and its capacity of penetration is inversely proportional to the extent of the wave. The smaller the electromagnetic wavelength, the greater its capacity to penetrate the different materials and organic tissues.

The detection of infrared radiation is possible using cameras and special apparatuses that have such specific sensitivity. This technology is widely used by several industrial segments for the thermal control of equipment, for the preventive maintenance of components. The war industry also produces equipments that detect infrared radiation in order to detect soldiers in places with low or no light

Specific equipment have currently been developed capable of detecting infrared radiation produced by the human body in a non-invasive manner, and to demonstrate functional changes of several types of diseases, especially those of the circulatory system and painful syndromes, whose metabolic changes determine modifications in the local production of heat.

Thus, infrared radiation has physical properties that make it penetrate into the several organic tissues, in addition to having a great capacity to interact with carbon dioxide.

5—Organic Effects of Light

The effects of light on human beings are well known, yet little is known at this time regarding its direct and indirect therapeutic effects. As an example, after vitamin D is absorbed by the Digestive System, it is activated by the sunlight and allows calcium to be deposited into bone tissues, maintaining their normal density or delaying the natural process of osteoporosis.

The effects of visible light on the different tissues as a therapeutic modality is usually represented by therapies offered to icteric newborns (jaundice, high levels circulating bilirubin) and for the treatment of several dermatoses, for example, psoriasis, by means of ultraviolet light.

Many of the biological effects of the different visible light spectra and wavelengths, however, are still unknown, and are part of a new study area referred to as biophotomodulation.

Therefore, if infrared radiation came from a visible light source, it is very likely that the expected effects could be extended or a lot more specific, allowing a greater individualization of proposed therapies.

6—Organic Effects of Temperature

The effects of thermal changes in the human body are extremely important, altering systemic metabolic processes, contributing to or inhibiting enzyme reactions, as well as modifying actions of the immune system. The best example of this is the great importance of fever as an element of defense in the human body.

CO₂ is commercially available compressed in gas cylinders under high pressure, a fact that determines low temperatures of the gas. When it crosses the vascular system and the tubing of the gas injection apparatus, it gains heat, but still reaches the parenteral injection sites at temperatures well below the human physiological body temperature.

The presence of the drug “cold” in the tissues can cause inherent pain caused by the presence of the drug at the site. Distention of tissues at the infusion sites may also stimulate and produce pain.

In anatomical terms, there are cutaneous receptors for several physical and mechanical stimuli: heat, cold, pressure, pain, touch. The thermal and pain stimuli ascend to the central nervous system through a common medullary tract, referred to as thermalgic pathway.

The interpretation and judgment of temperature by the central nervous system are always made comparatively to its changes on the skin, whereas the hot-cold contrast is very important to augment or minimize the transmitted sensations.

It has also been shown that the skin has eight times more receptors for cold than for heat, and that these are very similar to the pain receptor fibers.

Focal thermal stimulus can trigger pain stimuli above 45 degrees Celsius at heating, and below 15 degrees Celsius at cooling. Triggering of cutaneous mechanoreceptors is also minimized when there is heating of the skin.

The main mechanism likely to reduce pain during parenteral CO₂ infusion caused by infrared radiation could be secondary to heating the skin and subcutaneous tissue determined by this radiation, which could complicate the triggering at free nerve endings (pain receptors) by increased triggering of heat receptor fibers (thermoreceptors).

Another likely mechanism for analgesia of the infrared radiation application sites would be to diminish the triggering of secondary mechanoreceptors to tissue heating, which are usually stimulated following the infusion of the gas due to the increased volume in body tissues.

Heating of the gas within the tissues after it has absorbed the infrared radiation could minimize the hot-cold contrast caused by its presence.

As to the CO₂ volume expansion by increasing the temperature within the 10 tissues, this will take place gradually and without compromising the count or velocity of the infusion. There could also be a more uniform distribution in the application areas, without producing painful stimuli due to the sudden expansion of tissues.

The analgesic and therapeutic effects that heating can produce in different body tissues are also well known: improvement of healing, infection control, pain relief, improvement of the movement amplitude of joints, among others.

Aside from direct cell biostimulation as a result of infrared radiation, the body tissues at the irradiated areas could also benefit from the secondary effects determined by the increased local temperature.

7—Body Thermometry

In order to maintain body temperature constant (homeothermia), humans produce heat that is usually lost to the environment. The main body structure that accomplishes this interface is the skin, representing several underlying organic structures, which belong to the same functional dermatome.

Skin microcirculation experiences constant variations provoked by motor fibers of the sympathetic autonomic nervous system, which produces skin vasoconstriction and vasodilatation as one of its main body temperature balance mechanisms, thermoregulation.

Still regarding body temperature, ischemic tissues often show reduced temperature, whilst inflammatory tissues, or those with higher vascularization, have increased temperature. Nonetheless, these changes are barely perceptible on normal physical exam. This is due to the fact that normal thermal sensitivity of the human hands is only capable of distinguishing, through direct palpation, variations greater than 02 (two) degrees Celsius.

Body thermometry through the skin can be obtained by several methods, using direct and indirect sensors. The more sensitive indirect sensors described have infrared radiation receptors, with a sensitivity of up to 0.02 degrees Celsius per square millimeter, which even allow the detection of long waves of the infrared spectrum (7.5 to 13 micrometers).

Currently, for higher precision thermometries, there are two modalities of high sensitivity infrared sensors: the FDA (Focal Plane Array), and the QWIP (Quantum Well Infrared Photodetector). Thus, the cutaneous temperature variations that are measured by these equipments and subjected to digital processing produce high resolution qualitative and high sensitivity quantitative graphical representations (images).

This patterns of temperature variation, imperceptible to sight or palpation, are assessed for distribution, form, symmetry as compared to the opposite side, and dynamic responses produced by several stimuli, such as the infusion of carbon dioxide, for instance.

Measurement of temperature variations at the parenteral injection sites of CO₂ would produce changes in cutaneous temperature that, when measured and processed in an appropriate software system, would control both the velocity and the volume of gas injected, adjusting it to a previously established temperature, and adjusting itself almost instantly to the individual variations of each tissue.

The intensity and wavelength of the infrared radiation focalized at the injection sites on the tissues can be controlled in the same way.

8—Medical Grade CO2 Infusion Controller Apparatuses

Carbon dioxide, also referred to as carbonic acid gas or carbonic anhydride, as a therapeutic medicinal product, is supplied as a USP form, and commercialized under compression in gas cylinders.

Because it is a non flammable, non toxic, and non emboligenic agent, it is the most commonly used gas in infusion apparatuses for performing video-laparoscopic surgeries. Its function in such surgeries is to create and maintain the pneumoperitoneum, a technique that distends the abdominal cavity, separating organs and allowing handling of organs under the gaze of cameras.

New CO₂ infusion apparatuses have been recently developed to perform the controlled subcutaneous injection of CO₂. In essence, they have a valve system that controls, under direct manual programming of the apparatus, either analogically or digitally, the volume and velocity of gas infusion. There is no previously described system for the control of infusion by direct or indirect body temperature.

The clinical application of such apparatuses has been directed to cosmetic and aesthetic changes, in which the injections of the gas would produce reduced localized fat, improved aspect of the skin, and reduced facial expression lines and dermal flaccidity. Its use in the treatment of “cellulite” (edematous fibrosclerotic panniculopathy) has also been well documented.

Recently, with the development of various models and the greater access to these types of CO₂ infuser equipment, their therapeutic of CO₂ could be expanded to various areas of medical expertise.

9—Therapeutic Light Emitting Apparatuses

Light appears in several wave dimensions, both in its visible spectrum as the invisible one. Thus, it has the possibility to produce several infrared wavelengths. Different infrared wavelengths produce different types of biointeraction, either cellular or modular to the activity of different tissues.

In an historical summary of the use of medical grade carbon dioxide of the innovaiton of the clinical applications, around 1930, in France, the therapeutic benefits of bathing in waters rich in carbon dioxide (CO₂) were identified, by improving the walking distance of patients that had arterial circulatory disease with intermittent claudication. The gas penetrated into the human body by direct diffusion through the skin (41), characterizing the transcutaneous injection of the drug. Soon after, at the same location, the subcutaneous injection of the gas was attempted in some patients; however, due to the lack of adequate technology at the time and to complications of this method, it was discouraged.

As regards Vascular Surgery and Angiology, carbon dioxide has also been widely used in the form of intra-arterial injections for performing angiographies in patients allergic to iodine contrast media, with no significant adverse reactions even at large volumes, demonstrating the biosafety of CO₂ when used as a drug.

Later in the 80's in Italy, a CO₂ infusion controller apparatus was developed with technological improvements that would allow control of the flow and volume of the gas, and also contained specific filters that made the injected gas practically sterile, making safe injections of the gas into the skin and subcutaneous tissue possible.

As a consequence, treatments were initiated using hypodermic injection of CO₂ directly into the affected areas. A new phase of use would, however, become a reality, intended for the area of medical aesthetics, particularly for the treatment of “cellulite” (edematous fibrosclerotic panniculopathy), localized fat, and skin flaccidity.

Recently, in Brazil, national apparatuses have been developed similar to the original Italian model, of which the first registration at the Brazilian National Health Surveillance Agency—ANVISA happened approximately two years ago. After that, new registrations were issued to other companies that also manufacture carbon dioxide controller apparatuses.

Such equipment were widely promoted and spread commercially to physicians of several specialties who practice aesthetic procedures, in the area referred to as “Aesthetic Medicine”, where its use has increasingly been shown to be extremely safe.

At this time, there are various CO₂ infusion controller apparatuses available in the market (Carbomed®, Carboxiderm®, Carbitron®, Carbitek®, CarboxExxpert®, Carboxide®), all of which, however, produce pain in response to the infusion of the gas, have no mechanisms that would promote its interaction with infrared radiation, or even have their CO₂ infusion controls determined by body thermometry.

There are also no reports or known equipment that would control the intensity of infrared radiation emission on the skin through cutaneous body thermometry, both by direct and indirect means.

### SUMMARY OF THE INVENTION

Following extensive analysis of the studies, practices, and equipment available to this day, the applicant, whose main activity is in the areas of Angiology and Vascular Surgery, has conducted extensive research and understood that direct thermal heating of the gas within the apparatus could cause it to expand its volume within the vascular system, making it difficult to control the flow and volume of the injection of CO₂.

Hence, the present proposal modifies the primary concept of thermally heating the gas, offering an alternative so that such heating may take place through the emission of infrared radiation, where CO₂ can absorb the radiation and be heated in a controlled manner, not only at the level of the apparatus, and conducting systems, but especially at the level of the injection sites, already within the organic tissues. This new alternative for the heating of CO₂ is much more attractive and safe, in addition to combining the other biological effects described below.

Another point observed by the applicant is the fact that, up to this date, no carbon dioxide (CO₂) applications have been described in several alternative parenteral routes, such as: intramuscular, intrasynovial, perineural, peritendinous, intralesional, perilesional, perifascial, periarticular, intrasynovial, perivascular, intravascular, retroperitoneal, peridural, subdural, and perineural.

Similarly, the applicant has also observed that there is no current description as to the pharmacodynamic properties of CO₂ infused via parenteral routes on the circulatory system: effects on microcirculation, induction of neovascularization, endothelial function stimulation, potentiation of collateral circulatory network formation, mechanisms of action on ischemic tissues or those subjected to venous hypertension, or even to chronic lymphatic stasis. There is also no description of the effects of such administration routes on peripheral nerves, acupuncture, and painful syndromes.

Similarly, the beneficial effects observed with the association of infrared radiation concomitantly with CO₂ with the intention to produce physical, chemical, and biological interactions, acting as a potentiating, promoting, or inducing agent of organic effects during the processes of parenteral injection of the drug have also not been described.

At present, there is no knowledge of any medical grade CO₂ infusion controller apparatus, either national or imported, that has the additional feature of concomitant emission of infrared radiation coming from thermal and/or light sources.

There is also no description as to the control of the parenteral infusion of CO₂, or even the intensity of the infrared radiation emission of being controlled by means of body thermometry, as can be seen in the apparatuses described above, which belong to the state of the art.

With the intention to verify the proposal in question, a national CO₂ injection controller apparatus was acquired, and medical work by the proponent has been initiated.

Based on the existing medical scientific knowledge, it is known that certain gases, such as O₂ (oxygen), O₃ (ozone), and NO (nitric acid), among other effects, can act as “messengers” in microcirculation, promoting vasoconstriction and vasodilatation in distinct tissues.

Thus, the applicant, based on the thought that CO₂ (carbon dioxide) may act directly both on the diameter of vessels, as well as in the intimacy of cell membranes in a way yet to be defined, perhaps on specific receptors, he began orientating his observations.

Due to his clinical experience, acquired from years of practice in the areas of Angiology and Vascular Surgery, particularly in the treatment of various painful syndromes, the applicant has noted that some patients with vascular and painful morbidities, who had been receiving treatment for body image disturbances (“aesthetics”), showed improvement of vascular and painful symptoms following injections of the gas.

It was perceived that the beneficial therapeutic effects in the areas of diffusion of CO₂ after its injection subcutaneously and intradermally explored the initial “aesthetic” objectives. As related to vascular diseases and painful syndromes, these “secondary” effects were shown to be highly desirable, with the improvement of many associated signs and symptoms.

Based on these clinical findings, and with the consent of said patients, as well as with the help of a number of volunteers, the applicant modified the sites and forms of parenteral injection of CO₂, moving them to areas closer to the painful morbidities and the circulatory system, and there was a clear intensification of the initially observed therapeutic effects.

The applicant also observed that the use of infrared radiation concomitantly with the parenteral injection of CO₂ produced an important analgesia during the infusion of the gas. The intensity of radiation, however, could cause skin lesions; thus, during the infusions, he started to use as a safety parameter the assessment of skin temperature on the sites were irradiation was applied.

Another important factor to be considered is that, when parenterally injected, carbon dioxide is rapidly eliminated from the sites of application, transported via blood flow and returned to the environment through breathing. In this way, if infrared radiation is not applied concomitantly with the parenteral injection of the gas, synergy of methods does not take place. In other words, in case these are applied at different times, the tissue will only benefit from the individual effects of each method.

The interfaces between the areas of knowledge and technology applied to lo health care are increasing and the medical knowledge has shown great advancements in recent years, especially due to the increase in diagnostic resources and the development of new therapeutic alternatives, both pharmacological and surgical.

The vast segmentation and specialization of knowledge leads to the frequent need for discussions on new alternatives for products that already have proven applications on human health, saving time and financial investments. The capacity, however, to come up with new applications requires knowledge and specific insight of the person who redirected a known therapeutic modality to absolutely innovative applications.

Descriptions on the mechanisms of action and the responses elicited by the different tissues and cells of the human body when exposed to direct injection of CO₂ still require further studies. The scientific descriptions indexed so far are very few and refer rather to the effects determined by the “baths” with water rich in CO₂ (45), involving one of the parenteral forms of application (subdermal), or describe only its effects on the fat tissue, with focus on the treatment of localized fat.

### BRIEF DESCRIPTION OF THE DRAWINGS

FIG. 1 is a schematic view of one embodiment of the apparatus.

FIG. 2 is a schematic view of another embodiment of the apparatus.

FIG. 3 is a schematic view of another embodiment of the apparatus.

To complement the present description in order to obtain a better understanding of the characteristics of the present invention, and according to a preferred practical embodiment of said invention, a set of drawing, herein attached, accompanies the description, in which the following is represented in an exemplified illustrative, although not limiting, manner:

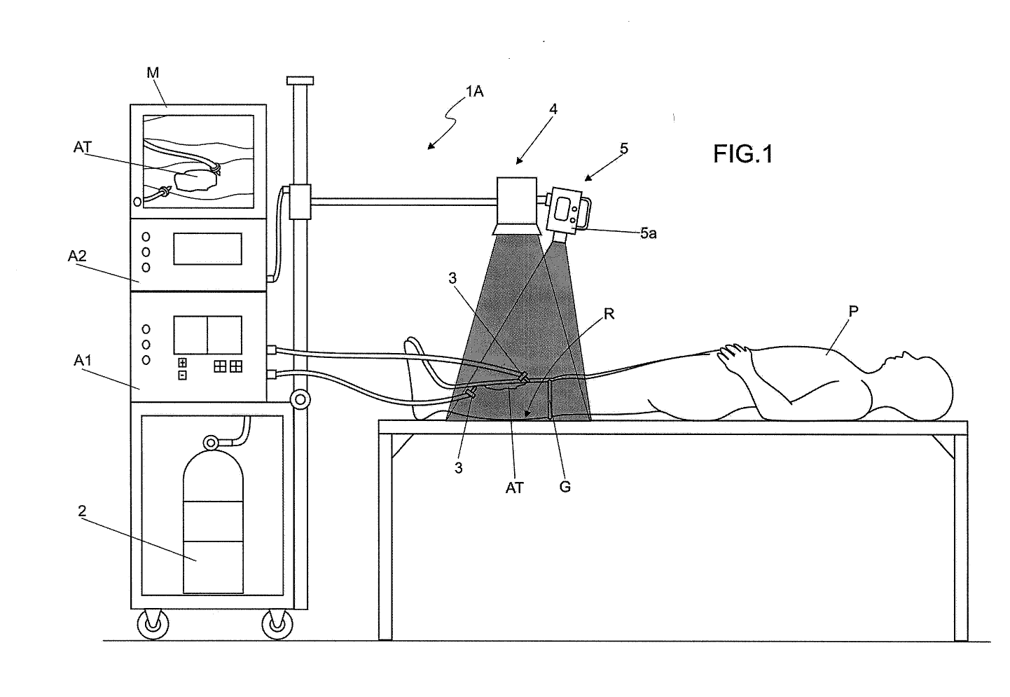

FIG. 1 schematically illustrates a complete apparatus that applies the method presently innovated, i.e., an apparatus that associates a control apparatus of CO₂ infusion through parenteral injection(s), with concurrent application of infrared radiation, of which the amount, flow, and velocity of CO₂ infusion, as well as the intensity and time of exposure of the treated area to the infrared radiation are controlled by an interface with an indirect cutaneous body thermometry apparatus, of the kind with infrared detection camera.

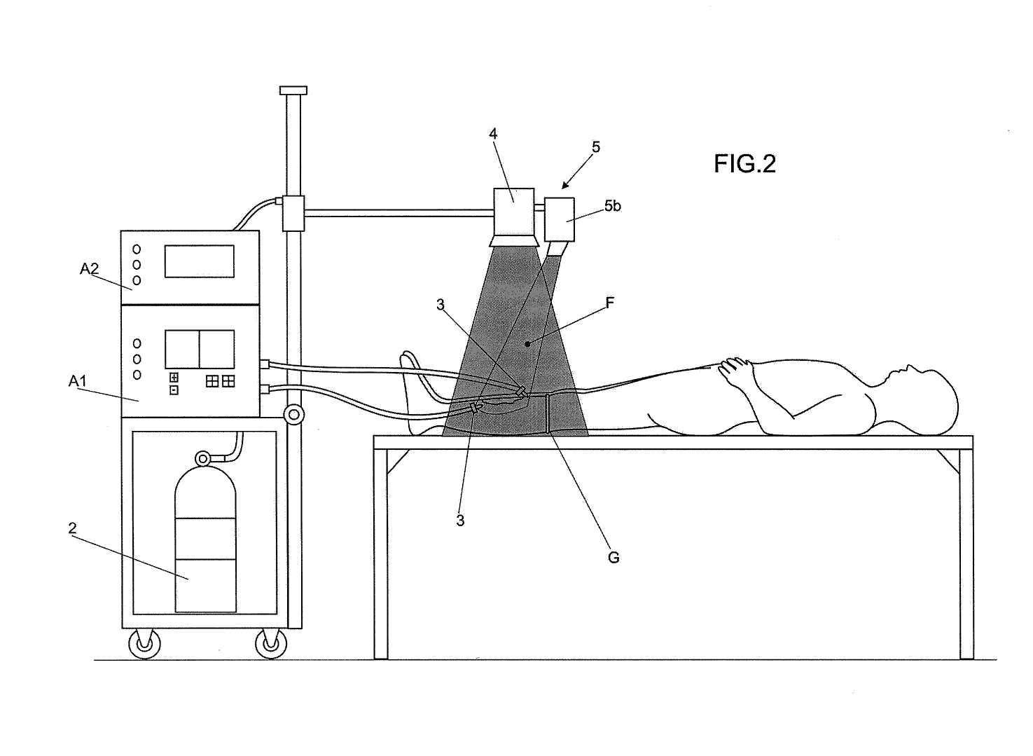

FIG. 2 schematically represents a variant of the apparatus shown in the previous figure, where, in order to perform thermometry, the infrared camera has been replaced by a device of the non contact thermometer type (with an infrared ray sensor), the orientation and focal distance of application of which is obtained by means of Laser emission.

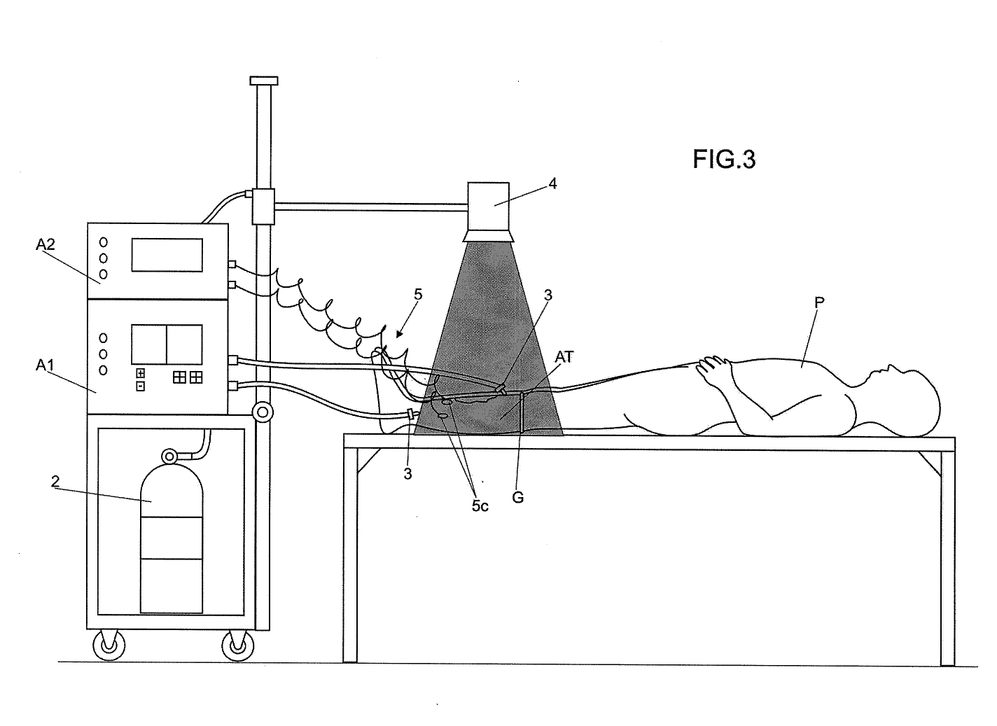

FIG. 3 schematically illustrates a more simplified apparatus that applies the method in question, for it anticipates an control and CO₂ infusion apparatus and another for the concurrent application of infrared radiation, in the same frames and functions already described in the previous figures, however, as opposed to them, the body temperature is measured by means of direct thermometry, obtained through skin sensors (thermal or infrared) applied directly over the treated area.

### DETAILED DESCRIPTION OF THE INVENTION

According to the illustrative figures described above, the present invention refers to a method and apparatus that associate the parenteral injection of medical grade carbon dioxide (CO₂) concomitantly with the application of infrared radiation from thermal and/or light sources using control by means of cutaneous and/or body thermometry, wherein, more precisely, the method is carried out by an apparatus (1), as presented in at least three versions (1A), (1B), and (1C) that associates means for the application of CO₂ (2) through parenteral injections with needle and filter (3), means for emitting and applying infrared light radiation (4), and means for controlling the CO₂ infusions, as well as the emission of infrared radiation by direct cutaneous thermometry (5) using a conventional contact thermometer (5d) or by a skin/contact infrared sensor (5c), or by indirect thermometry using non-contact thermometers (5b) with their focal orientation (F) obtained by a Laser (L) emitting apparatus, or yet by indirect thermometry obtained by infrared cameras (5a), with the principal intention to cause the association of infrared radiation to produce an interaction with the CO₂, acting in a way as to potentiate, promote, or induce organic effects, and as an agent that produces analgesia in a patient's (P) treated area (AT) during the processes of parenteral injection of the drug (3).

The control of the CO₂ infusion (2) into the organic tissues of the treated area (AT) is accomplished by an apparatus (A1) that acts concomitantly or concurrently with the apparatus (A2) that controls and determines the intensity of the infrared radiation emission by the respective source (4) in the treated areas (AT) in which the parenteral injection (3) of CO₂ (2) is applied.

The intensity of the infrared emission, as well as the amount/flow/pressure of the CO₂ infusion are to be controlled by cutaneous thermometry (5), which may present at least three forms of operation, which are:

a) (FIG. 1) indirect thermometry (5a) that comprises an infrared detection camera, capable of transmitting the image to a monitor (M), whilst at the same time it measures and transmits the caloric information of the area (R) were the injection of the CO₂ (2) and the infrared (4) are applied;

b) (FIG. 2) indirect thermometry (5b) that corresponds to a device that combines a thermometer that detects infrared with guided focus (F), as well as the focal distance obtained by means of a Laser (L) emitting apparatus that measures and transmits the calories to the apparatus (A2) that controls the injection of CO₂ (2) and infrared (4) in the treated area (AT); and

c) (FIG. 3) direct thermometry by infrared radiation receptors (5c) or conventional thermal receptors (5d), of the cutaneous sensor type applied over the skin of the patient around the treated area (AT), which measure and transmit the calories to the apparatus (A2) that controls the application of infrared (4) and CO₂ (2).

Technical variants in the mode of application of the product have also been developed by the proponent, such as: the use of limiting devices or tourniquets (G) at several points of the lower limbs in order to limit the expansion of the gas; distribution and homogenization of the CO₂ in areas of interest by means of manual massages with oils, creams, or ointments; as well as the development of specific injection techniques into alternative parenteral routes (intramuscular, subfascial, intrasynovial, and other), as well as new methodologies of injection of the gas (carboxyacupuncture, sclerocarboxytherapy, and carboembolotherapy), all representing methods to optimize the effects and the use of the proposed apparatus (1).

The present developed method includes two main stages:

Stage I) Application of the medical grade carbon dioxide—CO₂—in vascular diseases and painful syndromes consists of the following stages:

a) Provision of a limiting device (G) (adjustable tourniquet cuff made of latex or similar material) in order to prevent the proximal dispersal of the CO₂ gas (2) to lower and upper limbs, delimiting the anatomical area to be treated;

b) Cleaning of the entire area to receive the injections of the CO₂ gas (2) with 70° GL alcohol or another antiseptic solution;

c) Application of radiation to the area to be treated (AT) with a thermal or light source (4) infrared radiation emitting apparatus, controlled by body thermometry (5), prior to the initiation of the injection of the gas as per the technical description below;

d) Injections of the gas (2) via the parenteral route (3), standardized by the type of vasculopathy, painful syndrome, or induction of focal reactive oxygen therapy, adjusted by the type of treatment, and automatically controlled by the interfaces of the apparatus (A2);

e) Hemostasis, using gauze bandages and digital compression of the application points;

f) After the end of the parenteral injections (3), antisepsis of the entire treated area should be performed again with the same antiseptic solution used at the beginning of the procedure;

g) An adjuvant oil, cream, or ointment (neutral or medicated) should be applied to the entire treated area, and if homogenization of the gas distribution is required, a manual massage should be performed on the entire treated area, in a proximal to distal direction;

h) Standard treatment of bleeding areas, if any (mechanical cleaning and debridement);

i) Dressing of bleeding areas with non-adhesive products;

j) Bandaging of extremities (compressive or just for protection, depending on the pathology involved).

Stage II) Application of infrared radiation consists of the following stages:

a.1) Following antisepsis and placement of limiting devices (G) (tourniquets), initiate the application of 02 to 03 minutes of infrared radiation in the area to be treated with thermal or light infrared radiation emitters (4), prior to initiating the injection of the CO₂ gas (2);

b.1) The focal distance of radiation is variable depending on the thermal output capacity of the emitting source (4), and it is adjusted by a Laser (L) emitting apparatus, and calculated by the images obtained with the infrared camera (5a), indirect thermometer (5b), or direct cutaneous sensors (5c and 5d);

c.1) Monitoring with the infrared emitting source (4) the treated areas (AT) that are receiving the injection of CO₂ gas (2), throughout the procedure; in the areas with skin lesions of ischemic etiology, more caution should be exercised when using this radiation.

d.1) Caution should be taken to avoid burns, instructing the patient to warn in case of heat intolerance or a burning sensation produced by the infrared radiation.

Control and regulation of the amount/flow/pressure, as well as the dispersal of the CO₂ gas (2) in the tissues of the treated area (AT) would occur through changes in body temperature at the skin surface measured by specific equipment (5). The infrared radiation source (4) would have the irradiated heat intensity, the infrared wavelengths, as well as the cutaneous temperature increase in the treated area (AT) measured by these devices (5) and controlled by an interface apparatus (A2). This new methodology makes the process much safer and a lot less painful.

The Applicant, after having furthered his studies, and particularly because he operates in the areas of Angiology and Vascular Surgery, has developed, in conjunction with the present invention regarding the association of CO₂ application concomitantly with infrared radiation controlled by body thermometry, a technical standardization for the several modalities of parenteral injections of medical grade carbon dioxide (CO₂). This methodology, developed in association with the present patent, is protected by another form of legal protection, and applies to the different vascular diseases, painful syndromes, and induction of focal reactive oxygen therapy.

Along these lines, the applicant has conjectured several clinical applications for using the method and controlled apparatuses of carbon dioxide infusion (A1) combined with the emission of infrared radiation (4) controlled by cutaneous/body thermometry (5) in an interface apparatus (A2) in vascular diseases, painful syndromes, lo treatment of osteoarticular tissues, and induction of focal reactive oxygen therapy. Following tests and observations, the applicant obtained the following results with the application of the present invention:

1) In Arterial Vascular Disease:
Promotes or induces vascular neoangiogenesis;
Enlarges the collateral circulatory network;
Augments the arterial vascularization of the muscles;
Treats intermittent claudication;
Increases the walking distance in claudicating patients;
Treats ischemic pains due to resting;
Accelerates or induces healing of ulcers of arterial etiology;
Reduces and delimits areas of necroses;
Reduces paresthesias secondary to ischemic processes;
Improves the quality of the skin and annexes in ischemic areas;
Improves the functional symptoms of microcirculation;
Improves and stabilizes digital circulation of extremities;

2) In Venous Vascular Disease:
Reduces symptoms of chronic venous stasis;
Improves pains and cramping of venous etiology;
Reduces edema of extremities;
Intensifies intramuscular venous connections;
Promotes increased venules;
Reduces the vascular capillary permeability;
Improves dermatofibrosis;
Improves fibroscierotic retractions and cicatricial contractures;
Reduces stasis eczema and focal dermatitis;
Reduces the areas of hyperpigmentation (iron and melanin);
Augments vascularization of cicatricial areas;
Accelerates healing of ulcers in lower limbs;
Increases the motility of the ankle affected by dermatofibrosis;
Intensifies the efficiency of contraction of the calf (‘calf pump’);
Improves venous return;
Improves the myokinetic effect of lymphatics;

3) In Lymphatic Vascular Disease
Promotes and induces lymphangiogenesis;
Intensifies the pulsatile activity of lymphatic collectors;
Increases lymph flow;
Increases perivascular protein uptake;
Increases permeability of lymph capillaries;
Improves fibrosis of tissues with chronic inflammation;
Lymphogenesis in tissues with congenital lymphatic hypoplasia;
Lymphogenesis in tissues with secondary lymphatic lesion (lymphangitis);
Stimulates the muscle contractile activity of lymphatic collectors;
Reduces lipedemas;
Reduces lymphedemas;
Reduces fibroedemas;
Intensifies the effects of Complex Physical Therapy (Fold)

4) In Vascular Disease—Miscellaneous:
Accelerates the cicatricial process in lesions difficult to heal;
Accelerates the cicatricial process in areas of circulatory deficit;
a) Diabetic feet and plantar perforating disease ulcers;
b) Healing of pressure sores or scabs;
Free implanted skin grafts;
Augments donor areas of free skin grafts;
Circulatory improvement in advancement or myocutaneous flaps;

5) In Vascular Disease—Intramuscular therapies:
Sclerocarboxytherapy—Use of Sclerosants in combination with CO₂ foam (Escierocarboxi—Carboxifoam).
Embolizing substances combined with carbon dioxide gas foam for endovascular embolotherapies (Embolocarboxi—Carboxifoam).

6) In Painful Syndromes:
Several arthralgias in large joints;
Several arthralgias in small joints;
Lesions for repetitive efforts (LRE or work related osteomuscular diseases);
Extra-articular inflammatory processes;
Intra-articular inflammatory processes;
Painful processes by muscular contractures;
Fibromyalgias—‘trigger points’;
Inflammatory and ischemic neuralgias;
Degenerative neuritis;
Perineural circulatory stabilization;

7) In Acupuncture (Carboxyacupuncture);
Greater local effect at systemic and cranial acupuncture points;
Greater action spectrum on less sensitive meridians;
Greater longevity of treatment;

8) In direct therapies on bone tissues and related connective tissues;
Adjuvant in the treatment of osteomyelitis;
Adjuvant in the preparation bone graft recipient beds;
Adjuvant in the preparation of tendons and ligaments for grafting;
Adjuvant in the repair of ligament and tendinous lesions;
Facilitates intraoperatory dissection of tendinous sheaths;
Minimizes post tenolysis adhesions;

9) Focal reactive oxygen therapy (Effect similar to hyperbaric O₂)
Severe infections in soft tissues;
Adjuvant in the treatment of osteomyelitis;
Adjuvant in the treatment of pyoarthritis;
Aiding in the cicatricial process of extensive lesions;
Aiding in the repair of lesions in diabetics;
Aiding in the growth of granulation tissues;

It is important to understand that the present invention does not limit its application to the details and stages described herein. The invention is capable of other modalities, and of being practiced or executed in its variety of modes, and it is understood that the terminology is not limiting, but used for descriptive purposes.

### BIBLIOGRAPHY

1—Noções de Microcirculação. In: Mello N A, editor. Angiologia. 1^{a} ed. Rio de Janeiro: Guanabara Koogan; 1998. p. 29-41.
2—Boisseau, M. R.; Régulation vasomotrice de la microcirculation. In: Vayssairat, M.; Carpentier, P. eds. Microcirculation clinique. Paris: Masson, 1996. p. 40-70.
3—Yamashita, K.; Tochihara, Y. Effects of hyperoxia on thermoregulatory responses during feet immersion to hot water in humans. J Physiol Anthropol AppI Hum Sci, 2003; 22: 181-85.
4—Folberg, R.; Hendrix, M. J.; Maniotis, A. J.: Vasculogenic mimicry and tumor angiogenesis. Am J Pathol 2000;156 (2): 361-81.
5—Marshall, J. M. Chemoreceptors and cardiovascular control in acute and chronic systemic hypoxia. Braz J Med Biol Res, 1998, 31(7): 863-888.
6—Kawasuji, M.; Nagamine, H.; Ikeda, M.; Sakakibara, N.; Takemura, H.; Fujii, S S.; et. al. Therapeutic angiogenesis with intramyocardial administration of basic fibroblast growth facto. Ann Thorac Surg 2000; 69: 1.155-61.
7—Wagatsuma, S.; Konno, R.; Sato, S.; Yajima, A. Tumor angiogenesis, hepatocyte growth factor, and c-Met expression in endometrial carcinoma. Cancer 1998; 82:520-30.
8—Sant'Anna, R. T.; Kalil, R. A. K.; Moreno, P.; Anflor, L. C. J.; Correa, D. L. C.; et al. Gene therapy with VEGF 165 for angiogenesis in experimental acute myocardial infarction. Rev Bras Cir Cardiovasc 2003; 18(2): 142-147.
9—Lee, L. Y.; Patel, S. R.; Hackett, N. R.; Mack, C. A.; Polce, D. R.; El-Sawy, T.; et al. Focal angiogen therapy using intramyocardial delivery of an adenovirus vector coding for vascular endothelial growth factor 121. Ann Thorac Surg 2000; 69:14-24.
10—Speck, N. M. G.; Focchi, J. A.; Alves, A. C.; Osório, C. A. B.; Baracat, E. C. The relationship between endometrial adenocarcinoma staging and angiogenesis. Rev Bras Ginecol Obstet 2003. 25(6): 396-401.
11—Takeshita, S.; Zheng, L. P.; Brogi, E.; Kearney, M.; Pu, L. Q.; et al. Therapeutic angiogenesis. A single intraarterial bolus of vascular endothelial growth factor augments revascularization in a rabbit ischemic hind limb model. J Clin Invest 1994; 93: 662-670.
12—Griffioen, A. W.; Molema, G. Angiogenesis: potentials for pharmacologic intervention in the treatment of cancer, cardiovascular diseases, and chronic inflammation. Pharmacol Rev June 2000; 52(2): 237-68.
13—Lee, T. M.; Su, S. F.; Tsai, C. H.; Lee, Y. T.; Wang, S. S. Differential effects of cilostazol and pentoxifylline on vascular endothelial growth factor in patients with intermittent claudication. Clin Sci (Lond) September 2001; 101(3): 305-11.
14—Silverthorn, D. U. Comunicaçcão, integração e homeostase. In: Fisiologia humana: uma abordagem integrada. Editora Manole. São Paulo, Brasil. Segunda Edição, 2005. Pg 153-179.
15—Silverthorn, D. U. O metabolismo celular. In: Fisiologia humana: uma abordagem integrada. Editora Manole. São Paulo, Brasil. Segunda Edição, 2005. Pg 74-99.
16—Silverthorn, D. U. Fisiologia Respiratória. In: Fisiologia humana: uma abordagem integrada. Editora Manole. São Paulo, Brasil. Segunda Edição, 2005. Pg 497-535.
17—Tsui, J. C. S.; Baker, D. M.; Biecker, E.; Shaw, S.; Dashwood, M. R. Potential role of endothelin 1 in ischaemia-induced angiogenesis in critical leg ischaemia. Br J Surg 2002, 89 (6): 741-47.
18—Pinto-Coelho, R. M. Fotossíntese. (Pinto-Coelho R M ed) In: Fundamentos em ecologia. Artmed, Porto Alegre, 1^{a} Ed., 2002. Pp 159-65.
19—Pinto-Coelho, R. M. A energia solar na biosfera. (Pinto-Coelho R M ed) In: Fundamentos em ecologia. Artmed, Porto Alegre, 1^{a} Ed., 2002. Pp 139-145.
20—Keeling, C. D. Atmospheric CO₂ concentrations—Mauna Loa Observatory, Haway 1958-1956 NDP-001?RI Carbon Dioxide Information Center, Oak Ridge National Laboratory, Tenesse, USA.
21—Michlovitz, S. L. Biophysical principles of heating and superficial heating agents. In: Thermal Agents in Rehabilitation. (Michlovitz S L ed) Philadelphia: Davis, 1996; pp. 99-118.
22—Kitchen, S. S.; Partridge, C. J. Infra-red therapy. Physiotherapy 1991; 77: 249-254.
23—Jones, B. F. A reappraisal of the use of infrared thermal image analysis in medicine. IEEE Trans Med Imaging 1998; 17:1019-27.
24—Ring, E. F. J. Thermal symmetry of human skin temperature distribution Thermology Int, 1999; 9(2): 53-55.
25—Silverthorn, D. U. Introdução ao sistema endócrino. In: Fisiologia humana: uma abordagem integrada. Editora Manole. São Paulo, Brasil. Segunda Edição, 2005. Pg 186-208.
26—Hasson, S. M.; Williams, J. H.; Gadberry, W.; Henrich, T. Viewing low and high wave length light: Effect on EMG activity and force production during maximal voluntary handgrip contraction. Physiotherapy Canada, 1989; 41: 32-5.
27—Westerhof, W.; Siddiqui, A. H.; Cormane, R. H.; Scholten, A. Infrared hyperthermia and psoriasis. Arch Dermatol Res 1987; 279: 209-10.
28—Cervero, F.; Gilbert, R.; Hammond, R. G.; Tanner, J. Development of secondary hyperalgesia following non-painful thermal stimulation of the skin: a psychophysical study in man. Pain, 1993; 54(2): 181-9.
29—Fox, R. H.; Woodward, P. M.; Exton-Smith, A. N.; Green, M. F.; et al. Body Temperatures in the Elderly: A National Study of Physiological, Social, and Environmental Conditions. Br Med J, 1973; 27; 1: 200-6.
30—Hardy, J. D. Temperature regulation, exposure to heat and cold, and effects of hypothermia. In: Therapeutic Heat and Cold, (Lehmann J F, ed.). Baltimore: Williams & Wilkins, 1982; pp. 172-178.
31—Lehmann, J. F.; Masock, A. J.; Warren, C. G.; Koblanski, J. N. Effect of therapeutic temperatures on tendon extensibility. Arch Phys Med Rehabil 1970; 51(8): 481-7.
32—Lehmann, J. F.; De Lateur, B. J. Therapeutic heat. In: Therapeutic Heat and Cold, (Lehmann J F, ed.). Baltimore: Williams & Wilkins, 1990; pp. 404-562.
33—Uematsu S. Telethermography in the differential diagnosis of reflex sympathetic dystrophy and chronic pain syndrome. In: Rizzi R, Vinsentin M. Pain Therapy. New York: Elsevier Biomedical Press; 1983.
34—Burihan, E. O Exame Vascular. Suplencia Vascular (São Paulo) 2001; 2(7): 5-7.
35—Chan, F. H.; So, A. T.; Lam, F. K. Generation of three-dimensional medical thermograms. Biomed Mater Eng 1996; 6: 415-28.
36—Anbar, M. Computerized thermography. The emergence of a new diagnostic imaging modality. Int J Technol Assess Health Care 1987; 3(4):613-21.
37—Armstrong, D. G.; Lavery, L. A. Predicting neuropathic ulceration with infrared dermal thermometry. J Am Podiatr Med Assoc 1997; 87(7): 336-7.
38—Vilos, G. A.; Vilos, A. G. Safe laparoscopic entry guided by veress needle CO2 insufflation pressure. Am Assoc Gynecol Laparosc 2003; 10(3):415-20.
39—Ezio Belotti, Mario de Bernardi. Utilizzazione della. CO2 termale nella pannicolopatia edemato-fibrosclerotica. Rivista Italiana di Medicina Estetica 1992; No 2.
40—Wehr, T. A.; Rosenthall, N. E.; Sack, D. A. Role of light in the cause and treatment of seasonal depression. Photochem Photobiol 41 (suppl), 45.
41—Fabry, R.; Dubost, J. J.; Schmidt, J.; Body, J.; Schaff, G.; Baguet J. C. [Thermal treatment in arterial diseases: an expensive placebo or an effective therapy?] [Article in French] Therapie 1995; 50(2): 113-22.
42—Lang, E. V.; Gossler, A. A.; Fick, L. J.; Barnhart W.; Lacey, D. L. Carbon dioxide angiography: effect of injection parameters on bolus configuration. J Vasc Interv Radiol 1999; 10(1): 41-9.
43—Brandi, C.; D'Aniello, C.; Grimaldi, L.; Bosi, B.; Dei, l. Lattarulo, P.; Alessandrini, C. Carbon Dioxide Therapy in the Treatment of Localized Adiposities: Clinical Study and Histopathological Correlations. Aesthetic Plast Surg; May-June 2001; 25(3): 170-4.
44—Ferrara, N. Vascular endothelial growth factor: basic science and clinical progress. Endocrine reviews 2004; 25 (4): 581-611.
45—Irie, H., Tatsumi, T.; Takamiya, M.; Zen, K.; et. al. Carbon dioxide-rich water bathing enhances collateral blood flow in ischemic hindlimb via mobilization of endothelial progenitor cells and activation of No-cGMP system. Circulation 2005; 111: 1523-1529.

1. An apparatus for associating parenteral application of medical grade carbon dioxide gas concomitantly with application of infrared light radiation to an area to be treated of a target, the apparatus comprising:
(a) a source of infrared light radiation selected from the group consisting of: thermal source, light source and combinations thereof;
(b) means for applying the infrared light radiation to the area to treated;
(c) a source of medical grade carbon dioxide gas;
(d) means for concomitantly applying the medical grade carbon dioxide gas through parenteral injection to the area to treated;
(e) means for concomitantly controlling the carbon dioxide gas application and the infrared light radiation application through cutaneous and/or body direct and/or indirect thermometry.

2. The apparatus according to claim 1, wherein the means for applying the medical grade carbon dioxide gas through parenteral injections to the area to treated includes a especific tube connected to the source of medical grade carbon dioxide gas at a first end and to a especifics needles and a especific filter at a second opposite end, the opposite end placed adjacent to the area to treated.

3. The apparatus according to claim 1, wherein the cutaneous and/or body thermometry is a direct thermometry selected from the group consisting of: conventional contact thermometer and a skin-contact infrared sensor; or indirect thermometry selected from the group consisting of: a device combining an infrared detecting thermometer with guided focus obtained by a laser emitting device capable of measuring the focal distance; and an infrared camera capable of measuring the caloric information of the target and concomitantly measuring the infrared rays and of transmitting both measurements to a monitor.

4. A method for associating the parenteral injection of medical grade carbon dioxide gas concomitantly with the application of infrared light radiation to an area to treated of a target, and with controlled thermometry of the target, the method comprising the steps of:
(i) providing a limiting device constructed and arranged to prevent a proximal dispersal of the carbon dioxide gas to lower and upper limbs of the target or other bodies areas of the target, if delimiting the area to be treated of the target is necessary;
(ii) desinfecting the area to be treated with an antiseptic solution;
(iii) applying infrared light radiation from a source of infrared light radiation selected from the group consisting of: thermal source, light source and combinations thereof; to the area to be treated for a pre-determined period of time sufficient to reach a pre-determined temperature to reduce the pain produced by application of medical carbon dioxide gas via a parenteral route;
(iv) applying the carbon dioxide gas via a parenteral route, obtained biological and celular direct and/or indirect local and/or sistemic effects and reproducing the local effects obtained with hyperbaric oxygen therapy methods;
(v) concomitantly monitoring the carbon dioxide gas application and the infrared radiation application through cutaneous and/or body thermometry while performing steps (i) to (iv), optimizing the local analgesic effects and to prevent excessive thermal effects or burns;
(vi) hemostasy the area to be treated;
(vii) complementary desinfecting the area to be treated with the antiseptic solution;
(viii) applying a composition selected from the group consisting of: adjuvant oil, suitable cream, neutral ointment and medicated ointment, to the area to be treated;
(ix) manually massaging the treated area to homogenization of the gas into the tissues and/or proximal and/or distal distribution;
(x) further treating injured and/or bleeding areas by any means known in the art, if necessary;
(xi) dressing the injured and/or bleeding areas with non-adhesive products, if necessary;
(xii) bandaging lower and upper limbs, or other body areas, if necessary

5. The method according to claim 4, wherein the limiting device is an especific equipment, for example, an adjustable tourniquet.

6. (canceled)

7. The method according to claim 4, wherein in the step of applying the carbon dioxide gas via a parenteral route, the parenteral route is selected from the group consisting of: intramuscular, perifascial, peritendinous, periarticular, intrasynovial, perivascular, intravascular, tangential transcutaneous, superficial transcutaneous, deep transcutaneous, intralesional, perilesional, retroperitoneal, peridural, intradural, and perineural.

8. The method according to claim 4, wherein the step of applying the carbon dioxide gas is performed through a modality selected from the group consisting of: acupuncture procedures using needles emitting medical grade carbon dioxide gas, vascular esclerotherapy using medical grade carbon dioxide gas, and intravascular use of medical grade carbon dioxide gas to adjuntive embolotherapy ou oclusive endovascular procedures

9-22. (canceled)

23. The method according to claim 4, wherein objective comparative data obtained with direct and/or indirect thermometry is used as an objective data recording element or as a subsidiary comparative test for pre- and post-treatments with medical carbon dioxide gas infusion in parenteral routes and/or infrared radiation therapy assessments.

24-25. (canceled)

26. The method according to claim 4, further including the step of applying a source of infrared light radiation selected from the group consisting of: thermal source, light source and combinations thereof an analgesic agent onto the area to be treated during the step of injecting the carbon dioxide gas via parenteral injection.

27. (canceled)

28. The method according to claim 4, wherein the target present vascular diseases and/or painful syndromes and/or musculo-esqueletal and ostheoarticular diseases.

29. A method for applying carbon dioxide gas to a target via especific parenteral route, the parenteral route selected from the group consisting of: intramuscular, perifascial, peritendinous, periarticular, intrasynovial, perivascular, intravascular, tangential transcutaneous, superficial transcutaneous, deep transcutaneous, intralesional, perilesional, retroperitoneal, peridural, intradural, and perineural.

30. The method according to claim 29, wherein the method is performed through a modality selected from the group consisting of: acupuncture procedures using needles emitting medical grade carbon dioxide gas, vascular esclerotherapy using medical grade carbon dioxide gas, and intravascular use of medical grade carbon dioxide gas to adjuntive embolotherapy ou oclusive endovascular procedures.

31. A method for controlling the application of infrared light radiation to an area to treated of a target with controlled thermometry of the target, the method comprising the steps of:
(A) applying infrared light radiation from a source of infrared light radiation selected from the group consisting of: thermal source, light source and combinations thereof; to the area to be treated for a pre-determined period of time sufficient to reach a pre-determined temperature to obtain analgesic effects minimizing the pain of application of medical carbon dioxide bas by parenteral route;
(B) applying carbon dioxide gas via a parenteral route, to obtained biological and celular direct and/or indirect local and/or sistemic effects and reproducing the local effects obtained with hyperbaric oxygen therapy methods, and
(C) concomitantly monitoring the carbon dioxide gas application and the infrared radiation application through cutaneous and/or body thermometry while performing steps (A) and (B), to optimizing the local analgesic effects and to prevent excessive thermal effects and/or burns; wherein the cutaneous and/or body thermometry is a direct thermometry selected from the group consisting of: conventional contact thermometer and a skin-contact infrared sensor; or indirect thermometry selected from the group consisting of: a device combining an infrared detecting thermometer with guided focus obtained by a laser emitting device capable of measuring the focal distance; and an infrared camera capable of measuring the caloric information of the target and concomitantly measuring the infrared rays and of transmitting both measurements to a monitor.
